(19) United States
(12) Patent Application Publication
(10) Pub. No.: US 2026/0240671 A1
(43) Pub. Date: Aug. 20, 2026

(54) Medical Catheter with a Balloon Embedded in a Head

(71) Applicant: Zhejiang Baihuo Health Technology Co., Ltd., Shaoxing, Zhejiang (CN)

(72) Inventor: Dongsheng Dong, Beijing (CN)

(21) Appl. No.: 19/487,736

(22) PCT Filed: Mar. 27, 2024
(86) PCT No.: PCT/CN2024/084008

§ 371 Date: Nov. 25, 2025

(30) Foreign Application Priority Data
Mar. 25, 2023 (CN) 202310317120.3

(51) Int. Cl. A61F 2/962; A61F 2/958; A61M 25/00; A61M 25/06

(52) U.S. Cl. CPC A61F 2/962; A61F 2002/9583; A61M 2025/0057; A61M 2025/006; A61M 2025/0062; A61M 2025/0681

(57) ABSTRACT

A medical catheter with a balloon embedded in a head comprises a catheter head, a catheter middle portion, a catheter tail and a balloon. The catheter head has a head recess. The balloon is composed of a balloon connecting portion and a balloon free portion. At least one part of the balloon free portion is inserted into the head recess, and a farthest endpoint of the balloon free portion is located in the head recess. A lower gap communicating with a lower gap fluid passage is defined between the inner surface of the balloon free portion and the head sidewall of the catheter head. The balloon chamber area, located in the head recess for containing a drug, is bag-like and has an opening toward an outside of the head recess. When an external fluid enters the lower gap fluid passage and then enters the lower gap, a volume of the lower gap will increase, and the external fluid will squeeze the inner surface to decrease a volume of the balloon chamber area; and the balloon chamber area can disappear when the balloon free portion completely bulges out from the head recess so as to release the drug contained in the balloon chamber area.

### TECHNICAL FIELD OF THE INVENTION

The present invention relates to a technical field of medical instrument products, and in particular to a medical catheter with a balloon embedded in a head.

### BACKGROUND OF THE INVENTION

As a kind of thin tubes made of medical-grade materials, medical catheters have a wide range of functions and can be inserted into the human body for disease treatment or surgery. Medical catheters can be inserted into body cavities, blood vessels, the brain or adipose tissues to assist in drainage, delivery of liquids or solids, placement of surgical instruments, etc. Certain specialized catheters can also be used for preclinical or clinical researches to sample biological components such as lipophilic and hydrophilic compounds, neurotransmitters, peptides and proteins, antibodies, nanoparticles and nanocarriers, enzymes and vesicles.

When placed at a particular part of a biological living body, the medical catheter can be used as a gastric tube, a urethral catheter, a sputum suction tube, a biliary drainage tube, a ventricular drainage tube, a vascular stenosis expanding tube, etc. When in use, all medical catheter need to be inserted into the natural orifice, blood vessel, pathological sinus or other tracts of the biological living body. In the process of advancing through natural orifices of the biological living body, the medical catheter often encounters resistance, including natural stenosis, pathological stenosis, stenosis caused by smooth muscle reflex contraction, etc. A lubricant is usually coated on the surface of the medical catheter before being inserted, but the lubricant is nearly depleted when the medical catheter reaches a stenotic area. As a result, serious shearing injury and even perforation will be caused to the organism tissue if the catheter head forcedly passes through the stenosis. Even in case of successful intubation, it is more prone to secondary microbial infection due to local artificial injury of the tissue. These problems cannot be solved by the existing medical catheters.

The optimal approach for the stenosis caused by pathological reasons such as scarring, edema or blood coagulation is to accurately release the lubricant, glucocorticoid, anaesthetic, anticoagulant and the like by the catheter head before the catheter passing through the stenosis. When it is useless to release the lubricant accurately if the catheter encounters a serious stenosis, it is necessary to extend the catheter head out of the balloon to dilate the tissue at the stenosis so as to facilitate passage.

The present invention provides a medical catheter having a balloon embedded in the catheter head, a drug can be contained in a balloon chamber, and the catheter head has a head recess, it can make the drug to be accurately released at the target position while the medical catheter smoothly passing through a natural orifice of a living body.

### SUMMARY OF THE INVENTION

For solving at least one above technical problem, the medical catheter comprises a catheter head, a catheter middle portion, a catheter tail; and a balloon which consists of a balloon connecting portion and a balloon free portion; wherein, the catheter head has a head recess for receiving the balloon free portion, and a head sidewall surrounding the head recess ; the balloon connecting portion is sealed to the catheter head, the balloon has an inner surface, an outer surface, a balloon chamber area enclosed by the outer surface, and a farthest endpoint; at least one part of the balloon free portion is inserted into the head recess, and the farthest endpoint of the balloon free portion is located in the head recess; a lower gap is defined between the inner surface of the balloon free portion and the head sidewall, a lower gap fluid passage communicates with the lower gap through a lower gap opening in the lower gap which communicates with an external fluid; the balloon chamber area, located in the head recess for containing a drug, is bag-like and has an opening toward an outside of the head recess; when an external fluid enters the lower gap fluid passage and then enters the lower gap, a volume of the lower gap will increase, and the external fluid will squeeze the inner surface to decrease a volume of the balloon chamber area; and, under the squeezing of the external fluid, the balloon chamber area disappears when the balloon free portion completely bulges out from the head recess so as to release the drug contained in the balloon chamber area.

To expand the balloon easily, preferably, the medical catheter further comprises a hollow elastic balloon sleeved on the catheter tail or the catheter middle portion near the catheter tail, the hollow elastic balloon has an internal space communicating with the lower gap fluid passage, and fluid in the internal space of the hollow elastic balloon can be injected into the lower gap by squeezing the hollow elastic balloon.

The way of the balloon connecting portion being sealed to the catheter head comprises the following solutions: the balloon connecting portion like a membrane-bag structure is sleeved outside the head sidewall in a sealed manner, and the balloon free portion is turned over and then embedded into the head recess of the catheter; or, the balloon connecting portion is directly sealed to the surface of the head recess, and the balloon free portion is directly embedded into or embedded into the head recess of the catheter after turning over.

The catheter head is generally composed of a separate head component and a catheter-head tube body connected with each other in a sealed manner. The balloon connecting portion can be directly sealed to the head component, or to the catheter-head tube body, or to both at the same time. When the separate head component is applied, the head recess is located within the head component. The head component and the catheter-head tube body can also be integrally molded as a single component.

The expression “at least a part of the balloon free portion is embedded in the head recess of the catheter head” means that the balloon free portion can be completely or partially embedded into the head recess of the catheter head.

The “farthest endpoint” of the balloon free portion refers to the point closest to a bottom of the head recess.

The gap between the inner surface of the balloon and the catheter head is defined as the lower gap, which means a space between the inner surface of the balloon and the outer head sidewall (surface) of the head component covered by the inner surface of the balloon. In specific implementations, a part of the head component or the whole head component can be covered by the inner surface of the balloon. a lower gap fluid passage communicates with the lower gap through a lower gap opening in the lower gap which communicates with an external fluid.

The lower gap opening in the lower gap communicating with an external fluid which makes the lower gap fluid passage communicate with the lower gap, means the lower gap opening locates on the surface of the catheter head covered by the balloon, which can be the sidewall of the head recess or the outer surface of the catheter head. The lower gap opening can directly communicate with the lower gap, or can communicate with the lower gap after passing through the balloon connecting portion.

At least a part of the balloon free portion is embedded into the head recess of the catheter head. This comprises two situations, i.e., the part of the balloon free portion is completely embedded into the head recess or is partially embedded into the head recess. When the balloon free portion is partially embedded, both a protruding portion of the outer surface of the balloon which is the part of the balloon free portion that bulges out from the head recess, and an embedded portion of the outer surface of the balloon which is embedded into the head recess form the balloon chamber area.

The balloon chamber area has an opening toward an outside of the head recess, means that the opening of the balloon chamber area is oriented directly above the top end of the catheter head. The opening of the balloon chamber area can also be oriented in a direction deviating from an angle directly above the top end of the catheter head as required, that is, the opening of the balloon chamber area can also be arranged obliquely.

The balloon chamber area disappears when the balloon free portion completely bulges out from the head recess, means that the balloon free portion embedded into the head recess is squeezed out from the head recess and bulges out from the head recess in a reverse direction, so that the bag-like balloon chamber area and the opening of the balloon chamber area can disappear completely at this time and the farthest endpoint of the balloon free portion is located outside the head recess. Due to different degrees of squeezing, the balloon free portion can partially bulge out from the head recess, so that the balloon chamber area in the head recess is correspondingly decreased, and the farthest endpoint of the balloon free portion can also be pushed out from the head recess at this time.

The release of the drug contained in the balloon chamber area can be achieved in the following situations: squeezing out, pushing out or ejecting the drug out. The drug can be in various dosage forms, which can be solid drugs such as fine granules, spheres or granules or can be gel, aqua, ointment or the like with different physical and chemical components. After the balloon free portion bulges out from the head recess, the outer surface of the inflated balloon can continuously push the drug outward. Drug release can be realized by pressuring the external fluid to enter the lower gap and then squeezing the inner surface of the balloon; or, the head component is directly squeezed by external compression, for example, being squeezed by the tissue when entering the stenosis of the natural orifice of a living body (e.g., a human body or an animal body), so that the drug is released from the opening of the balloon chamber area. The balloon can be made of non-compliant, partially-compliant or compliant materials. When the balloon is made of compliant material, it is particularly sufficient and safe for expansion at the stenosis of the natural orifice after the balloon is inflated.

To facilitate fluid drainage, preferably, the medical catheter has an inner chamber, an inner opening below the catheter head and an outer opening on the catheter tail; both the inner opening and the outer opening communicate with the inner chamber of the medical catheter.

Both the inner opening and the outer opening of the medical catheter are in fluid communication with the inner chamber of the medical catheter. That means, when entering into a natural orifice or other biological tissues, the inner opening can be used to drain urine in the bladder to the outside of the body (e.g., as an urethral catheter), suck sputum in the airway to the outside of the body (e.g., as a sputum suction tube), suck gastric juice to the outside of the body (e.g., as a gastrointestinal decompression tube), inject a nutrient solution into the stomach (e.g., as a nasogastric feeding tube), etc. With the design of the balloon embedded in the catheter head (head recess), the medical catheter can release lubricants, anaesthetics, pH regulating drugs, receptor blockers or agonists, chemotherapeutic drugs, antibacterial drugs, glucocorticoids and the like through the balloon in the catheter placement process, and after being inflated, the balloon can expand the natural orifice or other tracts of the living body at a narrow position to facilitate the medical catheter to pass through, for example, facilitating the medical catheter to pass through the nasopharynx, the hypertrophic prostatic part, the esophagus or the airway stenosis.

As another solution of the head recess of the catheter head, preferably, the head sidewall is lobe-shaped, and the head sidewall is composed of at least two sidewall lobes each having a top and a bottom; the top of each sidewall lobe is free, while the bottom of each sidewall lobe is connected to the bottom of adjacent sidewall lobes; a sidewall lobe gap is defined between two adjacent sidewall lobes; and, the free top of each sidewall lobe is located in the lower gap or outside the balloon. This design facilitates outward bulging of the balloon from the head recess when the medical catheter passes a narrow passage of the living body, so that the drug in the balloon chamber area is released, the balloon is expanded, and the sidewall lobe gap is allowed to contain drugs.

To avoid possible adhesion between the inner surface of the balloon and a surface of the head recess of the catheter head, preferably, the medical catheter further comprises an anti-adhesion mechanism and/or a filling medium; the anti-adhesion mechanism can be a rib or a groove, which is disposed on the inner surface of the balloon and/or the head recess; and, the filling medium can be pre-filled in the lower gap to partially or completely block contact between the inner surface of the balloon and the surface of the head recess, the filling medium can be selected from various types of lubricants, water, and even gas pre-filled in the lower gap.

To achieve a continuous injection of the drug, preferably, the balloon free portion has a thin wall point at which the balloon is easy to be damaged by an external force. When the lower gap is continuously filled with the external fluid (can be the drug) and the balloon is continuously expanded, the balloon is first damaged at the thin wall point.

Preferably, the thin wall point is arranged in a region near the farthest endpoint of the balloon free portion, and this region includes the farthest endpoint.

As an improved structure of the balloon, preferably, the balloon has a plurality of balloon bumps and/or balloon recesses disposed on the outer surface of the balloon.

The plurality of balloon bumps on the outer surface of the balloon are mainly distributed on the outer surface of the balloon free portion, and can be in the shape of points, needles, mastoids, fluffs, divergent lines, mazes or the like according to specific applications. The plurality of balloon recesses can be in the shape of round pits, irregular pits, suckers or the like. During manufacturing, the balloon bumps or the balloon recesses can be integrally molded with the balloon. The balloon bumps can be used to increase the strength of the balloon free portion, increase the friction with the biological tissue of the living body, increase the amount of the adhered drug, reinforce the stimulation to receptors of the human body, and the like. The balloon recesses can be used to increase the amount of the adhered drug and tract the attached tissue under negative pressure. The balloon bumps on the outer surface of the balloon can only be distributed in a region of the balloon free portion that constitutes the balloon chamber area, so that the catheter will not stimulate the inner surface of the natural orifice when passing through in the natural orifice of the living body; or, the balloon bumps can also be distributed in a region outside the balloon free portion that constitutes the balloon chamber area, to increase the degree of stimulation to the inner surface of the natural orifice, particularly the mucosal receptor. The balloon bumps and/or balloon recesses on the outer surface of the balloon can also be used to scrape and collect inflammatory secretions, blood clots or the like on the inner surface of the natural orifice.

To enable the bulged balloon free portion to return to the head recess again, preferably, an elastic vinculum having two ends is disposed in the head recess, a first end of the elastic vinculum is connected to the farthest endpoint of the balloon free portion or in a region adjacent to the farthest endpoint of the balloon free portion, and a second end of the elastic vinculum is connected to the catheter head in the head recess. When the external fluid injected into the lower gap flows out through the lower gap fluid passage, the retraction of the extended elastic vinculum can pull the balloon free portion bulging out from the head recess of the catheter back to the head recess of the catheter, and the catheter can be continuously pushed to scrape the mucosal tissue, secretion or the like of the natural orifice into the balloon chamber area to serve as a test sample.

To continuously expand the biological tissue at the stenosis (the narrow position) of the natural orifice, preferably, the medical catheter further comprises a fixed cuff and/or an expanding cuff below the catheter head; the expanding cuff has an inner chamber sleeved on the medical catheter, the medical catheter has a catheter body and an expanding cuff fluid passage in the catheter body, and the expanding cuff fluid passage communicates with the inner chamber of the expanding cuff, when the expanding cuff is disposed in a stenosis of a natural orifice that has been pre-expanded by the balloon, the expanding cuff inflates after the external fluid is injected into the expanding cuff fluid passage and enters into the inner chamber of the expanding cuff, so as to expand corresponding biological tissue at the stenosis of the natural orifice.

The fixed cuff and/or the expanding cuff may be one or more in number. The design of the expanding cuff can be used for the expansion and treatment of urethral cicatricial stricture, prostatic hyperplasia, ureterostenosis, biliary stricture, stenosis of fallopian tubes, local stenosis of airways at various levels and the like, and can also be used for the expansion of various surgical sinuses. The difference between this type of expansion of the present invention and the prior art is that the balloon-embedded structure of the head minimizes the shearing injury when the catheter head passes through the stenosis (the narrow position), and the subsequent expansion and treatment by the expanding cuff is carried out while maintaining the integrity of the internal surface of the natural orifice, achieving a minimal tissue injury.

To support the corresponding biological tissue at the stenosis, preferably, the medical catheter further comprises a stent inflation cuff having an inner chamber sleeved on the medical catheter below the catheter head, and a stent sleeved on the stent inflation cuff, the medical catheter has a catheter body and an stent inflation cuff fluid passage in the catheter body, and the stent inflation cuff fluid passage communicates with the inner chamber of the stent inflation cuff; when the stent inflation cuff is disposed in a stenosis of a natural orifice that has been pre-expanded by the balloon, the stent inflation cuff inflates after the external fluid is injected into the stent inflation cuff fluid passage in the catheter body and enters into the inner chamber of the stent inflation cuff, so as to expand the stent and expand corresponding biological tissue at the stenosis.

Preferably, the stent inflation cuff is made of a non-compliant material. The way of distracting the stent in the present invention differs from the prior art in that: in the prior art, generally, the expanding catheter is placed first to expand the biological tissue at the stenosis and then taken out, and the catheter sleeved with the stent is then placed within, with cumbersome steps; while in the present invention, when the catheter head passes through the biological tissue at the stenosis (the narrow position), since the balloon-embedded structure of the head can expand the biological tissue at the stenosis after the balloon is inflated, the shearing injury is minimized and the stent inflation cuff is inflated to directly expand the stent sleeved thereon, so that the two steps of placing the expanding catheter first and then the catheter sleeved with the stent in the prior art are completed in one step, the injury to the patient is reduced and the medical efficiency is improved.

The foregoing orientation description is based on the fact that the catheter head faces the sky and the catheter tail faces the floor, that is, the catheter head serves as the top end of the medical catheter and the catheter tail serves as the bottom end of the medical catheter. The terms “upper”, “top”, and “front” refer to directions toward the top end of the catheter, while “lower”, “bottom” and “rear” refer to directions toward the bottom end. The specific positional relationships are determined by the reference numerals in the drawings. When in use, the catheter head and the catheter middle portion usually enter into the natural orifice of the living body, while the catheter tail is located outside the natural orifice.

Compared with the prior art, the present invention has the following beneficial effects.
1. In the present invention, the balloon is inserted inside the head recess of the catheter head of the medical catheter, the balloon can contain various therapeutic drugs in different dosage forms, including solid granules, microcapsules, liquid preparations and the like, and the drugs can be released after the catheter head reaches the target position within the biological living body, thus achieving a precise drug delivery.
2. The balloon embedded in the catheter head of the medical catheter can bulge out from the head recess of the catheter head and come into close contact with the biological tissue, so that the drug released from the balloon chamber area can be fully coated onto and the penetration of the drug into the biological tissue is promoted by pressurization.
3. When the medical catheter encounters a narrow position of the natural orifice, blood vessel or other tracts of the living body during advancement, the head sidewall can be deformed toward (a center of) the head recess, so that the diameter of the catheter head can be reduced and be easy to pass through the stenosis (narrow position).
4. The balloon embedded in the catheter head of the medical catheter can contain the lubricating drug which can be released at the stenosis of the natural orifice, blood vessel or other tracts of the living body in the advancement of the medical catheter, thus achieving an accurate lubrication and allowing the catheter to smoothly pass through the stenosis.
5. When the balloon embedded in the catheter head of the medical catheter is inflated, the balloon can expand and squeeze the swollen tissue of the biological living body to reduce its local volume. When the biological tissue at the stenosis is expanded by the balloon, it is helpful for the medical catheter to pass through the stenosis after deflation.

6. When the medical catheter encounters a resistance during advancement, the balloon embedded in the catheter head of the medical catheter can be inflated to radially expand the biological tissues, and the medical catheter is pushed forward at the time when the orifice is temporarily widened, thus avoiding the shearing injury caused by the forceful insertion of the catheter head of the medical catheter, and reducing the risk of secondary infection caused by shearing injury, especially for a medical catheter that is required an indwelling period longer than 24 hours. The medical catheter of the present invention also provides an optimal natural orifice, blood vessel and other tract conditions for the placement of various stents, such as prostate stents or intravascular stents, which may need to be operated immediately after the advancement of the medical catheter.
7. After the balloon embedded in the catheter head of the medical catheter is inflated, the balloon can be restored due to the traction of the elastic vinculum and embedded into the head recess of the medical catheter again, and can be repeatedly inflated as required.
8. When the balloon embedded in the catheter head of the medical catheter is inflated, the plurality of balloon bumps and/or balloon recesses on the outer surface can enhance the intensity of stimulation to the biological tissue and increase the capacity of containing biological tissue samples.
9. When the medical catheter enters into a biological living body, the thin wall point of the balloon free portion embedded into the catheter head can be damaged by in-vitro operation. After the balloon barrier disappears, the drug can be fully infused from the outside of the body through the lower gap fluid passage, to overcome the shortage of the carried drugs caused by the limited space of the balloon chamber area.

### BRIEF DESCRIPTION OF THE DRAWINGS

For a clearer understanding of the technical schemes in the embodiments of the present invention, the drawings referenced in the descriptions of the embodiments are briefly introduced below. It should be understood that the drawings described below illustrate only some embodiments of the present invention, and that other drawings may be obtained by those skilled in the art without inventive effort.

FIG. 1A is a sectional view of a medical catheter according to Embodiment 1 of the present invention when main components of the medical catheter are separated (exploded);

FIG. 1B is a sectional view with an enlarged view of the medical catheter according to Embodiment 1 when components of the medical catheter are assembled;

FIG. 1C is a perspective view with an enlarged view of the medical catheter according to Embodiment 1 when parts of the medical catheter are omitted;

FIG. 1D is a schematic view showing a head recess of a catheter head according to Embodiment 1 reaching a stenosis of a natural orifice;

FIG. 1E is a schematic view showing partial liquid drug is released from a balloon chamber area according to Embodiment 1;

FIG. 1F is a schematic view showing complete liquid drug is released from the balloon chamber area according to Embodiment 1;

FIG. 1G is a schematic view showing a balloon free portion of the balloon passing through a stenosis of a natural orifice according to Embodiment 1;

FIG. 1H is a schematic view showing a part of the medical catheter with a solid drug contained inside the balloon chamber area according to Embodiment 1;

FIG. 1I is a schematic view showing the solid drug is released from the balloon chamber area according to Embodiment 1;

FIG. 2A is an exploded view with an enlarged view of a medical catheter according to Embodiment 2;

FIG. 2B is a perspective view with an enlarged view of a partial medical catheter showing an anti-adhesion mechanism disposed in a head recess according to Embodiment 2;

FIG. 2C is a perspective view with an enlarged view of a partial medical catheter showing a filling medium pre-filled in a lower gap according to Embodiment 2;

FIG. 2D is a sectional view of FIG. 2C;

FIG. 2E is a sectional view with an enlarged view of a partial medical catheter according to Embodiment 2 showing a lower gap opening on the head sidewall;

FIG. 3 is a sectional view with an enlarged view of a medical catheter according to Embodiment 3;

FIG. 4A is a sectional view with an enlarged view of a medical catheter according to Embodiment 4 when a balloon is in a non-expanded state;

FIG. 4B is a sectional view with an enlarged view of the medical catheter according to Embodiment 4 when the balloon is in an expanded state;

FIG. 5A is a sectional view with an enlarged view of a medical catheter according to Embodiment 5 when an elastic vinculum is in an unstretched state;

FIG. 5B is a sectional view with an enlarged view of the medical catheter according to Embodiment 5 when the elastic vinculum is in a stretched state;

FIG. 6A is a schematic view of a medical catheter according to Embodiment 6 showing a balloon passing through a stenosis;

FIG. 6B is a schematic view of a medical catheter with an expanding cuff according to Embodiment 6 showing a stenosis is expanded by the expanding cuff;

FIG. 6C is a perspective view of the medical catheter according to Embodiment 6;

FIG. 7A is a perspective view of a partial medical catheter according to Embodiment 7 when a stent inflation cuff is in an unexpanded state;

FIG. 7B is a perspective view of a partial medical catheter according to Embodiment 7 when the stent inflation cuff is in an expanded state;

FIG. 7C is a schematic view showing the expansion of biological tissue at a stenosis by the stent inflation cuff of the medical catheter according to Embodiment 7.

### DETAILED DESCRIPTION OF THE INVENTION

The present invention will be further described below in detail by embodiments with reference to the accompanying drawings.

### Embodiment 1

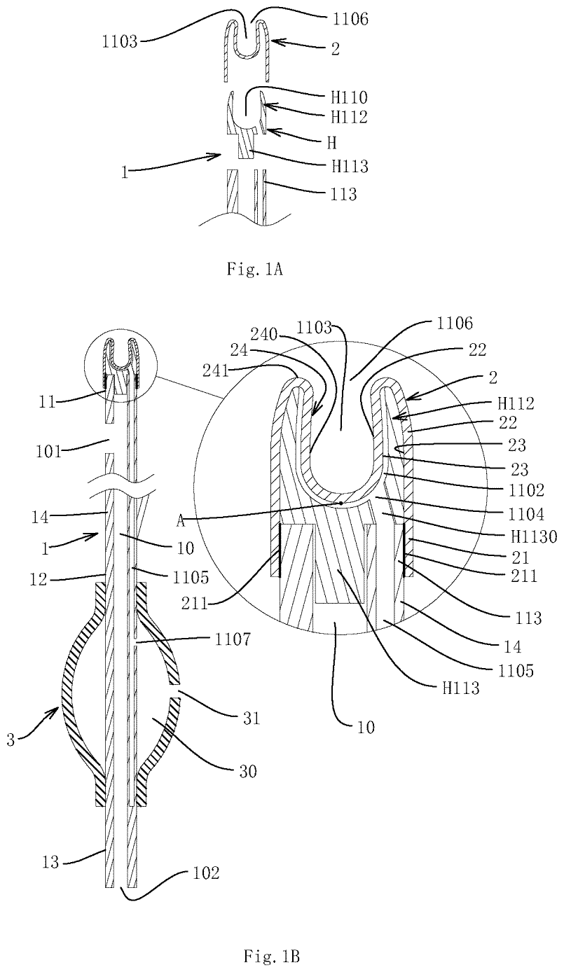

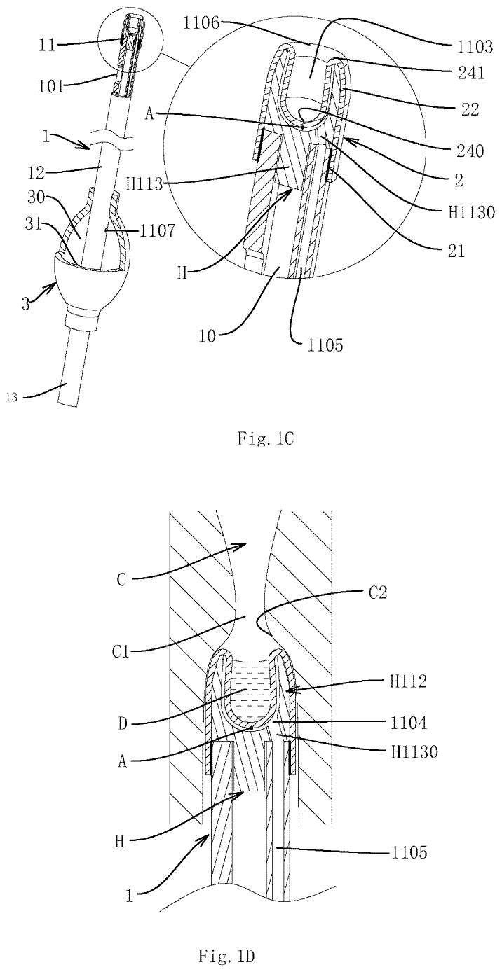

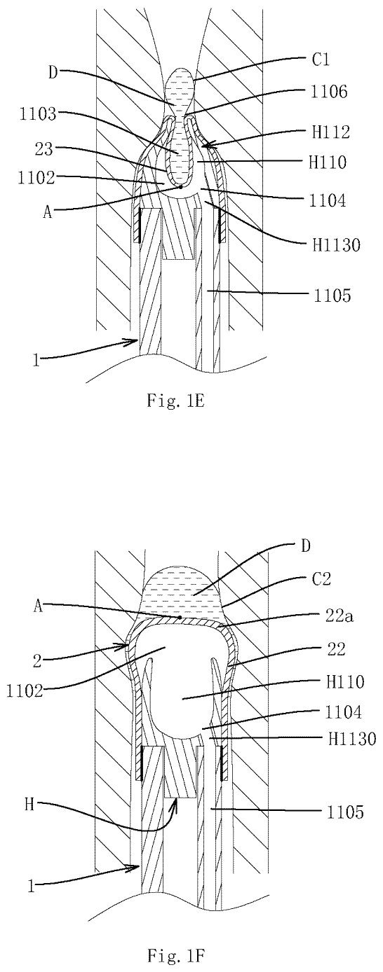

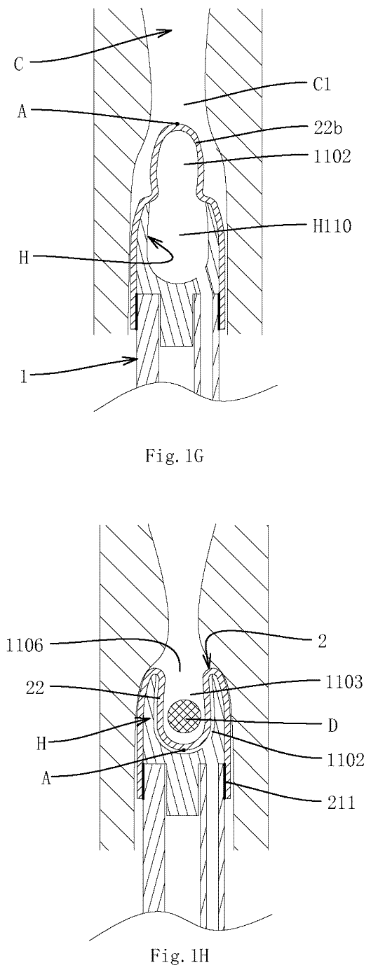

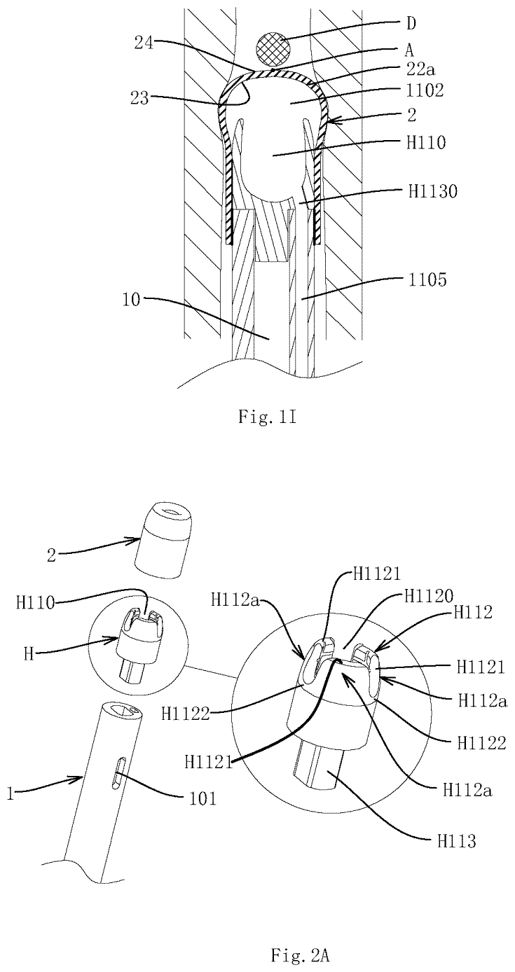

FIGS. 1A-1I show a first preferred embodiment of the medical catheter with a balloon embedded in a head of the present invention. The medical catheter 1 comprises a catheter head 11, a catheter middle portion 12 and a catheter tail 13, which form a catheter body. The catheter body is a hollow tube enclosed by an annular sidewall 14. The catheter head 11 has a catheter head tube 113 and is formed by a separate head component H preferably made of an elastic material sealedly connected to the catheter head tube 113. The hollow medical catheter 1 has an inner chamber 10, an inner opening 101 below the catheter head 11 and an outer opening 102 on the catheter tail 13. The head component H is composed of a head sidewall H112 extending upward and a bottom section H113 extending downward. The bottom section H113 of the head component H has a T-shaped section and is inserted into the inner chamber 10 of the medical catheter 1. The head component H has a recess area below a top of the head component H which is defined as a head recess H110 of the catheter head 11. The head component H has a head sidewall H112 surrounding the head recess H110. A bag-like thin-walled balloon 2 is connected to the catheter head 11, and is preferably made of a flexible material, e.g., silicone rubber or latex, and preferably has a wall thickness of less 0.5 mm, and is composed of a balloon connecting portion 21 and a balloon free portion 22. The balloon connecting portion 21 is sealed to the catheter head 11. In this embodiment, a region of the balloon connecting portion 21 which is circumferentially sealed to an outer surface of the catheter head tube 113 is defined as a tubular thin-layer sealing region 211, the sealed connection manner can be welding or bonding. The position of the balloon connecting portion 21 is fixed relative to the catheter head 11; while as the balloon free portion 22 does not adhere (be connected) to the catheter head 11, the balloon free portion 22 is easy to deform after being stressed. A portion of the balloon free portion 22 extending downward is embedded in the head recess H110 of the catheter 1, the balloon free portion 22 has a farthest endpoint A, and at least the farthest endpoint A of the balloon free portion 22 is located inside the head recess H110. The balloon 2 has an inner surface 23 and an outer surface 24. A lower gap 1102 is defined between the inner surface 23 of the balloon 2 and the catheter head 11, specifically, the lower gap 1102 refers to a gap between the inner surface 23 of the balloon 2 and an outer surface of the head sidewall H112 of the head component H covered by the inner surface 23 of the balloon 2. The volume of the lower gap 1102 is less than the volume of the head recess H110. In this embodiment, almost the whole head component H is covered by the inner surface 23 of the balloon 2. The head component H has a lower gap opening 1104 communicating with the lower gap 1102 so that an external fluid can flow into the lower gap 1102 through the lower gap opening 1104, the catheter 1 has a lower gap fluid passage 1105 communicating with the lower gap opening 1104. In this embodiment, the lower gap fluid passage 1105 is located at the annular sidewall 14 of the catheter 1. Since the balloon free portion 22 is inserted in the head recess H110 of the catheter 1, a bag-like space enclosed by the outer surface 24 acting as the boundary is defined as a balloon chamber area 1103. In this embodiment, the balloon free portion 22 has a protruding portion 241 located at the outer surface 24 of the balloon bulging out from the head recess H110, and an embedded portion 240 located at the outer surface 24 of the balloon embedded into the head recess H110. The balloon chamber area 1103 is jointly formed by the protruding portion 241 and the embedded portion 240. The balloon chamber area 1103 can be used to contain a drug D. FIGS. 1D-IF show that a liquid drug D is contained in the balloon chamber area 1103. The balloon chamber area 1103 has an opening 1106 on the top of the catheter head 11 toward outside the head recess H110. In this embodiment, for the convenience of containing the drug, the balloon chamber area 1103 is designed with a small opening 1106 and a large bottom, but it may also be designed with a large opening and a small bottom for easier molding during manufacture in other embodiments. A hollow elastic balloon 3 is sleeved on the catheter tail 13 or the catheter middle portion 12 near the catheter tail 13. The elastic balloon 3 has an inner opening 1107 on the outer sidewall of the catheter 1, an external opening 31 on a wall of the elastic balloon 3 and an internal space 30. The internal space 30 is communicated with the lower gap fluid passage 1105 through the inner opening 1107. By squeezing the elastic balloon 3, the fluid in the internal space 30 of the elastic balloon (usually, the air contained in the internal space 30) can be injected into the lower gap 1102.

When in use, as shown in FIGS. 1C-1D, when the medical catheter 1 advances in a natural orifice C of a biological living body and reaches a stenosis C1, the medical catheter 1 is squeezed by an inner surface C2 of the natural orifice, and the head sidewall H112 with a free state can be firstly deformed toward the head recess H110, so that the outer diameter of the catheter head 11 is reduced and it is easy to pass through the stenosis C1 (as shown in FIG. 1E). When the advancement of the medical catheter 1 in the natural orifice C encounters resistance, the external opening 31 of the elastic balloon 3 can be blocked by a finger of the user and the elastic balloon 3 can be squeezed at the same time by the user (not shown), the air in the inner space 30 will enter into the lower gap fluid passage 1105 through the inner opening 1107 and then enter into the lower gap 1102 through the lower gap opening 1104, accordingly the volume and pressure of the lower gap 1102 will be increased, the inner surface 23 of the balloon 2 is deformed under stress, and the volume of the balloon chamber area 1103 will be centripetally reduced. Thus, part of the liquid drug D contained in the balloon chamber area 1103 flows out from the opening 1106 of the balloon chamber area 1103 (as shown in FIG. 1E). The liquid drug D will be ejected when the elastic balloon 3 is squeezed rapidly and forcefully. When the pressure continues to increase, the balloon chamber area 1103 disappears since the balloon free portion 22 completely bulges out from the head recess H110 of the catheter 1, and the liquid drug D contained in the balloon chamber area 1103 will be completely released (as shown in FIG. 1F). At this time, the volume of the lower gap 1102 is greater than the volume of the head recess H110, and the bulged balloon free portion 22a can expand and squeeze the inner surface C2 of the natural orifice, accordingly the stenosis C1 of the natural orifice can be expanded. After the expanded state lasts for several seconds or longer, the elastic balloon 3 is released by the user, and the bulged balloon free portion 22a is retracted but usually cannot return into the head recess H110 and instead becomes a front-protruding balloon free portion 22b with a relaxed state as shown in FIG. 1G. At this time, since the expanded natural orifice C is not restored, the medical catheter 1 can be pushed to pass through the original stenosis successfully. When the stenosis is caused by the swollen biological tissue, this operation has the same effect.

The balloon chamber area 1103 also disappears since the balloon free portion 22 bulges out from the head recess H110 of the catheter 1, meaning that the balloon free portion 22 embedded in the head recess H110 is squeezed out from the head recess H110 and bulges out from the head recess H110 frontward, at this time the bag-like balloon chamber area 1103 and the opening 1106 of the balloon chamber area 1103 disappear completely and the farthest endpoint A of the balloon free portion 22 is located outside the head recess H110 (as shown in FIG. 1G). Due to different degrees of squeezing, the balloon free portion 22 may also partially bulge out from the head recess H110, accordingly the balloon chamber area 1103 is correspondingly decreased, and the farthest endpoint A of the balloon free portion 22 can also be pushed to move frontward but still be located inside the head recess H110 at this time (as shown in FIG. 1E).

When the balloon 2 has a relatively larger wall thickness, such as a wall thickness greater than 0.5 mm and a relatively short balloon chamber area 1103, the bulged balloon free portion 22 can return into the head recess H110 after retraction, but it will waste internal space and reduce the drug-carrying capacity of the balloon chamber area 1103 under such situation.

This embodiment can be applied to various medical catheters, such as gastric tubes, urethral catheters, sputum suction tubes, biliary drainage tubes and ventricular drainage tubes. Generally, gastric juice and urine flow into the inner chamber 10 through the inner opening 101 and are then discharged through the outer opening 102, and the liquid can also be infused in a reverse direction. By applying the technology of the present invention, if the above medical catheter encounters resistance when advancing in the natural orifice or other sinuses of the biological living body, the balloon 2 embedded into the catheter head 11 can be inflated to radially expand the biological tissue. During the temporary widening of the natural orifice C after expansion, the medical catheter is then pushed forward, thus avoiding the shearing injury to the channel tissue of the biological living body caused by the forced movement of the catheter head 11. This reduces the likelihood of secondary infection caused by shearing injury, particularly for medical catheters that must remain indwelling for more than 24 hours. This also creates optimal tract conditions for the placement of various stents such as airway stents, biliary stents, esophageal stents, intravascular stents, prostate stents or ureteral stents that is possibly started immediately after the placement of the medical catheter 1.

The balloon chamber area 1103 of the catheter head 11 can contain various therapeutic drugs D in different dosage forms, including solid granules, microcapsules, tablets in different shapes (including spherical), liquid preparations and the like, and releases the drugs after the catheter head 11 reaches the target position of the biological living body, thus realizing accurate drug delivery. After the drug D is released, the expanded balloon free portion 22a tightly contacts biological tissues, fully coating the drug released from the balloon chamber area 1103 onto the tissues and promoting penetration through applied pressure. When the balloon chamber area 1103 is used to contain a lubricant, it is more beneficial for the medical catheter to pass through the stenosis by releasing and coating the lubricant at the stenosis of the natural orifice C or the like and in combination with the expansion effect of the balloon. When a solid drug D is applied, the drug D can be further pushed forward.

The releasing operation of the drug D contained in the balloon chamber area 1103 includes squeezing, pushing or ejecting the drug D out. The solid drug D in FIGS. 1H-1I is spherical. The balloon chamber area 1103 can also contain solid drugs in different shapes, such as fine granules, cones or capsules, including slow release drugs, or can contain gel, aqua, ointment or the like with different physical and chemical components. After the balloon free portion 22 bulges out from the head recess H110, the outer surface 24 of the inflated balloon 2 continuously pushes the drug D outward. The releasing of the drug D can be realized by making the external fluid be pressed to enter into the lower gap 1102 and then squeezing the inner surface 23 of the balloon 2, or be realized by directly squeezing the head component H of the catheter 1 by an external force, for example, being squeezed by the tissue when entering the stenosis of the natural orifice of a living body (e.g., a human body or an animal body), accordingly the drug D is released from the opening 1106 of the balloon chamber area 1103. The balloon 2 can be made of non-compliant, partially-compliant or compliant materials. When the balloon 2 is compliant, for example, being made of silicone, latex, thermoplastic elastomer or other materials, it is particularly sufficient and safe for expansion at the stenosis of the natural orifice after the balloon 2 is inflated.

### Embodiment 2

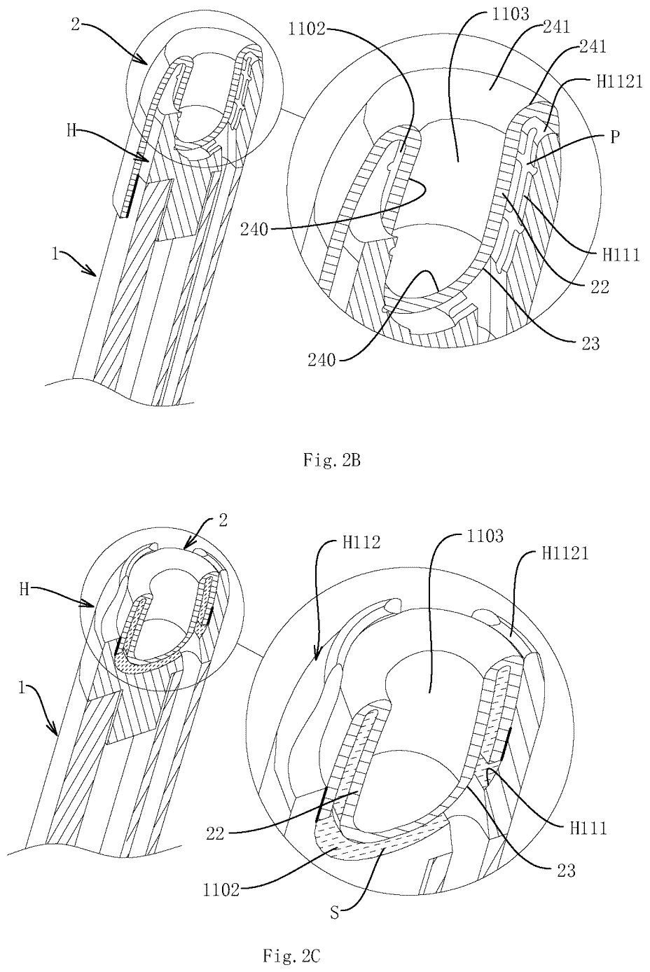

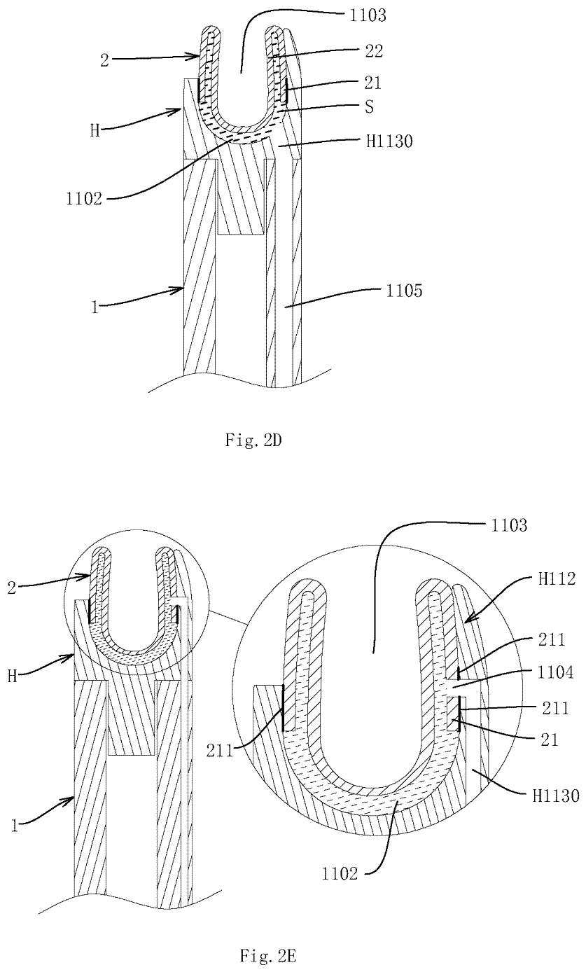

As shown in FIGS. 2A-2E, this embodiment differs from the above Embodiment 1 in that: the head sidewall H112 is lobe-shaped, and the head sidewall H112 is composed of three sidewall lobes H112a each having a top H1121 and a bottom; the top H1121 of each sidewall lobe is free, while the bottom of each sidewall lobe is connected to the bottom of adjacent sidewall lobes; a sidewall lobe gap H1120 is defined between two adjacent sidewall lobes H112a; and, the free top H1121 of each sidewall lobe is located in the lower gap 1102 or outside the balloon 2. As shown in FIG. 2B, the top H1121 of each sidewall lobe is located inside the lower gap 1102. The balloon free portion 22 is partially embedded in the head recess H110 of the catheter 1, and has a protruding portion 241 located at the outer surface 24 of the balloon bulging out from the head recess H110, and an embedded portion 240 located at the outer surface 24 of the balloon embedded into the head recess H110. A balloon chamber area 23 is jointly formed by the protruding portion 241 and the embedded portion 240. In the situations shown in FIGS. 2C-2D, the top H1121 of each sidewall lobe is located outside the lower gap 1102, and the balloon free portion 22 is completely embedded into the head recess H110. In this structure, the sidewall lobe gaps H1102 are exposed, and can contain the drug. For example, when there is a stenosis during advancement of the medical catheter in the natural orifice of the living body, it is easier to be squeezed and centripetally deformed to release the drug. Particularly, the lubricating drug makes it easier for the catheter to advance. Compared with the annual sidewall design in Embodiment 1, when the balloon 2 is activated, the tops H1121 of the sidewall lobes are easier to deform outward, and it is beneficial for the balloon 2 to eject out from the head recess H110. The drug contained in the balloon chamber area 1103 can be different from the drug contained in the sidewall lobe gaps H1120, so that the drugs can be flexibly applied according to a requirement in actual situation.

As shown in FIG. 2B, the catheter head 1 has an anti-adhesion mechanism P on the head recess H110 to avoid possible adhesion between the inner surface 23 of the balloon 2 and a surface H111 of the head recess H110 of the catheter 1. The anti-adhesion mechanism P can be ribs and grooves. In this embodiment, the anti-adhesion mechanism P is multiple crisscrossed grooves as shown in this figure. Thus, the contact area between the inner surface 23 of the balloon 2 and the surface H111 of the head recess H110 of the catheter 1 is reduced, and the space for filling the fluid is increased. Another anti-adhesion solution is shown in FIGS. 2C-2D, a filling medium S is pre-filled in the lower gap 1102 to partially or completely block contact between the inner surface 23 of the balloon 2 and the surface H111 of the head recess H110 of the catheter 1. The filling medium S can be selected from various types of lubricants, water, and even gas pre-filled in the lower gap 1102.

A variation of this solution, as shown in FIG. 2E, the lower gap opening 1104 is located in the tubular thin-layer sealing region 211, the lower gap opening 1104 communicates with H1130. The lower gap opening 1104 communicates with the lower gap 1102 after passing through the tubular thin-layer sealing region 211 of the balloon connecting portion 21.

### Embodiment 3

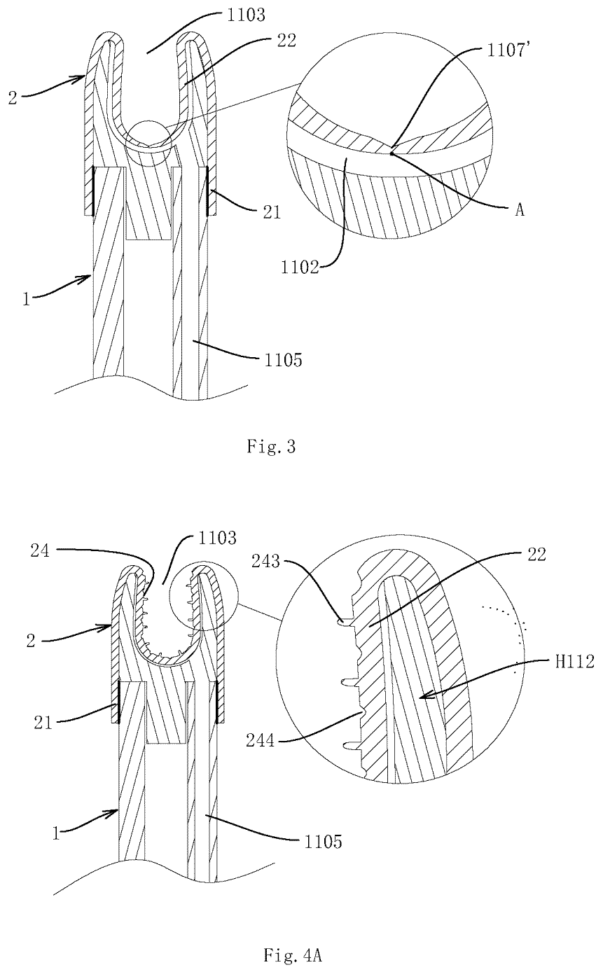

As shown in FIG. 3, this embodiment differs from Embodiment 1 in that: the balloon free portion 22 has a thin wall point 1107 at which the balloon 2 is easy to be damaged by an external force in a region near the farthest endpoint A of the balloon free portion 22. This thin wall point 1107 is formed by a weak connection, and at the thin wall point 1107 the wall thickness of the balloon 2 is small. When the lower gap 1102 is continuously filled with the fluid and continuously inflated, the thin wall point 1107 will be damaged and broken first, therefore the lower gap 1102 is communicated with the outside, and the drug can be continuously injected into the natural orifice or pathological sinus of the living body from the lower gap 1102 passing through the broken balloon 2, to overcome the shortage of the carried drug caused by the too small volume of the balloon chamber area 1103.

### Embodiment 4

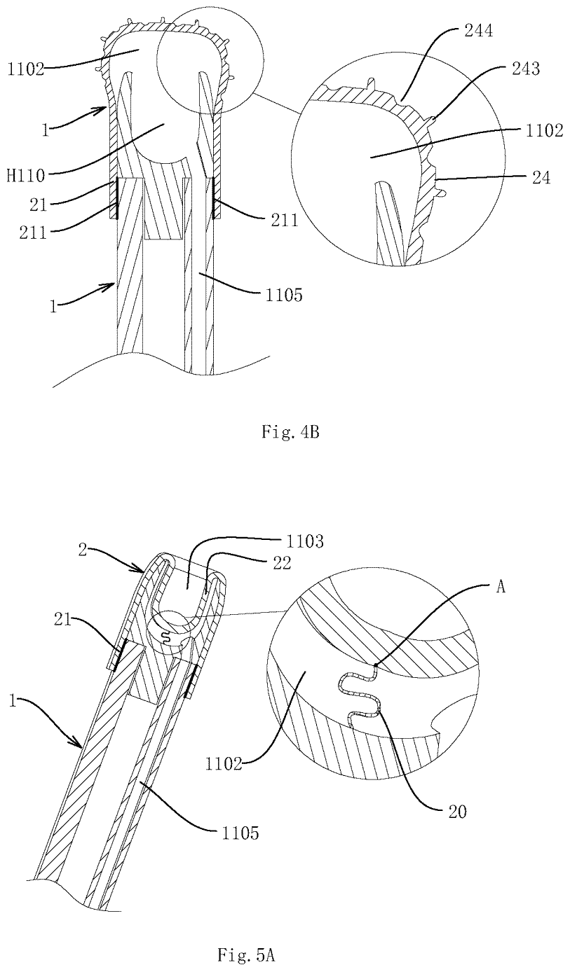

As shown in FIGS. 4A and 4B, this embodiment differs from Embodiment 1 in that: the balloon 2 has a plurality of balloon bumps 243 and balloon recesses 244 disposed on the outer surface 24 of the balloon 2. The balloon bump 243 is mastoid-shaped, and the balloon recess 244 is round-pit-shaped. The plurality of balloon bumps 243 and the balloon recesses 244 are mainly distributed on the outer surface of the balloon free portion 22, and are integrally molded with the balloon 2 during manufacturing. The balloon bumps 243 can be used to increase the strength of the balloon free portion 22, increase the friction with the biological tissue of the living body, increase the amount of the adhered drug, enhance the stimulation to receptors of the human body, and the like. The balloon recesses 244 can be used to increase the amount of the adhered drug and drag the attached tissue under negative pressure. In this embodiment, as shown in figures, the balloon bumps 243 are distributed only in a region of the balloon free portion 22 that constitutes the balloon chamber area 1103, so that the catheter 1 will not stimulate the inner surface of the natural orifice when advancing in the natural orifice of the living body. The balloon bumps 243 can also be distributed in a region outside the balloon free portion 22 that constitutes the balloon chamber area 1103, to increase the degree of stimulation to the inner surface of the natural orifice, particularly the mucosal receptor (not shown in figures). The balloon bumps 243 and the balloon recesses 244 can also be used to scrape off and contain inflammatory secretions, blood clots or the like on the inner surface of the natural orifice. FIG. 4A shows the balloon 2 before being inflated, and FIG. 4B shows the balloon 2 after being inflated.

### Embodiment 5

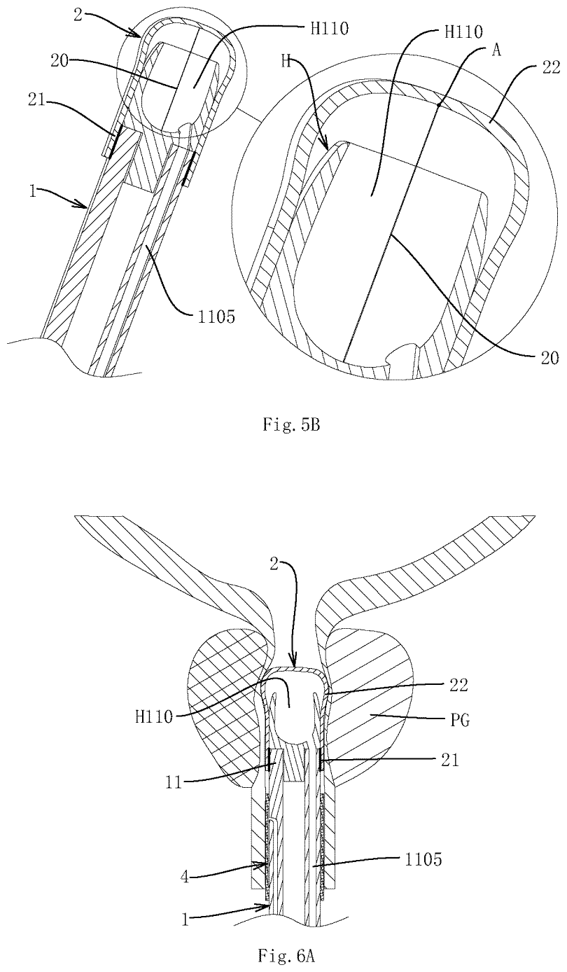

As shown in FIGS. 5A and 5B, this embodiment differs from Embodiment 1 in that: an elastic vinculum 20 having two ends is disposed in the head recess H110, a first end of the elastic vinculum 20 is connected to the farthest endpoint A of the balloon free portion 22 adjacent to the farthest endpoint A of the balloon free portion 22, and a second end of the elastic vinculum 20 is connected to the catheter head 11 in the head recess H110. As shown in FIG. 5B, when the balloon free portion 22 bulges out from the head recess H110, the elastic vinculum 20 is forced to stretch; and, when the fluid injected into the lower gap 1102 flows out through the lower gap fluid passage 1105, the elastic vinculum 20 contracts and pulls the balloon free portion 22, which has bulged out from the head recess H110 of the catheter 1, back to the head recess H110 of the catheter 1 (as shown in FIG. 5A), the balloon chamber area 1103 is restored to its original state, and the catheter 1 can be continuously pushed to scrape the mucosal tissue, secretion or the like of the natural orifice into the balloon chamber area 1103 to serve as a test sample. FIG. 5A shows the state that the elastic vinculum 20 is not stretched, and FIG. 5B shows the state that the elastic vinculum 20 is stretched.

### Embodiment 6

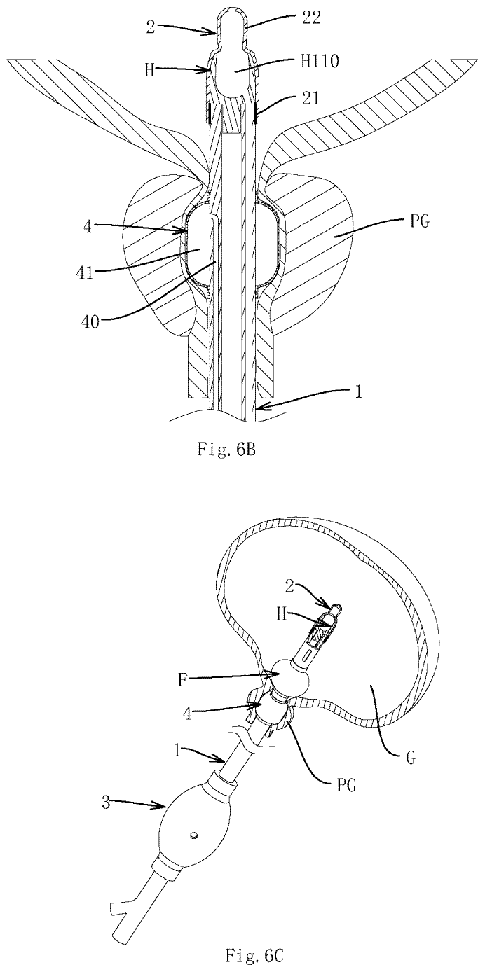

As shown in FIG. 6A, this embodiment differs from Embodiment 1 in that: the medical catheter further comprises an expanding cuff 4 having an inner chamber 41 sleeved on the medical catheter 1 below the catheter head 11, the medical catheter 1 has a catheter body and an expanding cuff fluid passage 40 in the catheter body, and the expanding cuff fluid passage 40 communicates with the inner chamber 41 of the expanding cuff 4. For example, when the advancing catheter head 11 encounters a urethral stenosis caused by prostate PG hyperplasia, the catheter head 11 can smoothly pass through the stenosis under the expansion of the balloon 2. As shown in FIG. 6B, when the expanding cuff 4 is disposed in a stenosis of a natural orifice that has been pre-expanded by the balloon 2, the expanding cuff 4 inflates after the external fluid is injected into the expanding cuff fluid passage 40 in the catheter body, and then enters the inner chamber 41 of the expanding cuff 4 to inflate the expanding cuff 4 so as to expand the corresponding biological tissue at the stenosis of the natural orifice. A fixed cuff F can be additionally provided on the basis of this embodiment. As shown in FIG. 6C, the fixed cuff F enters the cavity G of the bladder, is then inflated and stays at the bladder orifice to prevent the catheter head 11 from separating from the cavity G of the bladder. The expanding cuff 4 arranged below the fixed cuff F is located in the prostate of the urethra. After being inflated, the expanding cuff 4 can stably and continuously expand the prostate PG. The expanding cuff 4 can be made of a compliant material or a non-compliant material. When the medical catheter is used for the treatment of prostatic hyperplasia and the expanding cuff 4 is made of a non-compliant material, the maximum expandable diameter of the expanding cuff 4 is preferably the same as the maximum diameter of the existing urethral catheter, accordingly the injury possibly caused by expanding the urethra is minimized.

The expanding cuff 4 provided on the basis of the present invention can be used for the expansion and treatment of urethral cicatricial stricture, prostatic hyperplasia, ureterostenosis, biliary stricture, stenosis of fallopian tubes, local stenosis of airways at various levels and the like, and can also be used for the expansion of various surgical sinuses. This difference between the type of expansion and the prior art is that, the balloon-embedded structure of the catheter head of the present invention minimizes the shearing injury when the catheter head 11 passes through the stenosis, and the subsequent expansion and treatment by the expanding cuff 4 is carried out while maintaining the integrity of the internal surface of the natural orifice, achieving a minimal tissue injury.

### Embodiment 7

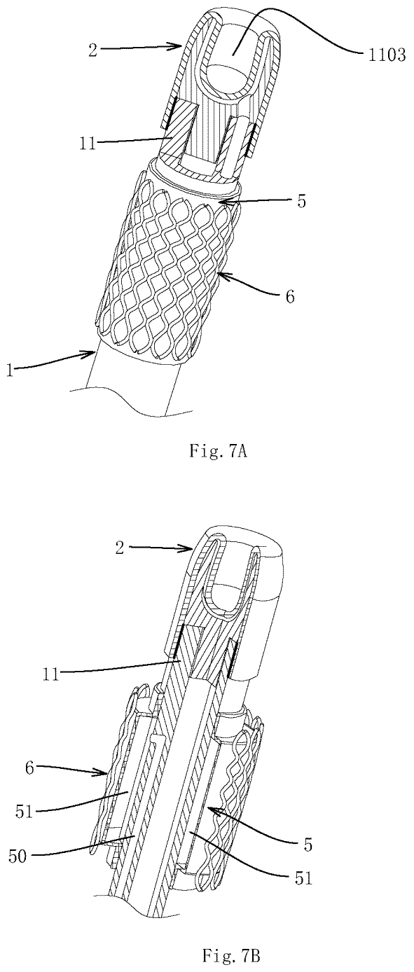

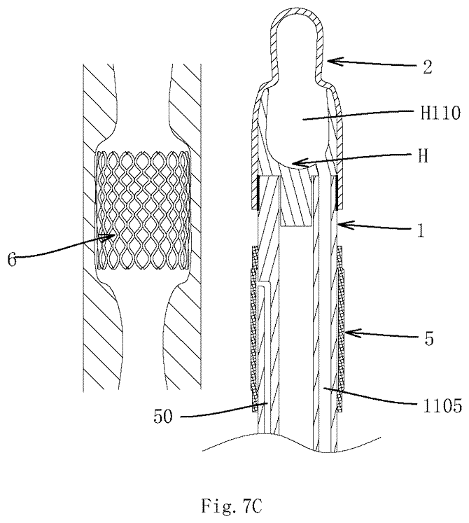

As shown in FIGS. 7A-7C, this embodiment differs from Embodiment 1 in that: the medical catheter further comprises a stent inflation cuff 5 having an inner chamber 51 sleeved on the medical catheter 1 below the catheter head 11, and a stent 6 sleeved on the stent inflation cuff 5, the medical catheter 1 has a catheter body and an stent inflation cuff fluid passage 50 in the catheter body, and the stent inflation cuff fluid passage 50 communicates with the inner chamber 51 of the stent inflation cuff 5. The stent inflation cuff 5 is preferably made of a non-compliant material. When the stent inflation cuff 5 is disposed in a stenosis of a natural orifice that has been pre-expanded by the balloon 2, the stent inflation cuff 5 inflates after the external fluid is injected into the stent inflation cuff fluid passage 50 in the catheter body and enters into the inner chamber 51 of the stent inflation cuff 5, so as to expand the stent 6 and expand corresponding biological tissue at the stenosis. FIG. 7A shows the state that the stent inflation cuff 5 is not expanded and the stent 6 is not distracted; FIG. 7B shows the state that the stent inflation cuff 5 is expanded and the stent 6 is expanded; and, FIG. 7C shows the state where the expanded stent 6 acts on the biological tissue at the stenosis after the stent inflation cuff 5 is taken out.

Compared with the prior art, the way of expanding the stent 6 has the following obvious advantages compared with current techniques. In the prior art, usually, the expanding catheter is placed first to expand the biological tissue at the stenosis and then taken out, and the catheter sleeved with the stent 6 is then inserted, resulting in complicated steps and secondary injury to the natural orifice or vascular endothelium. In the present invention, when the catheter head 11 passes through the biological tissue at the stenosis, since the balloon-embedded structure of the catheter head 11 can expand the biological tissue at the stenosis after the balloon 2 is inflated, the shearing injury is minimized, and the stent inflation cuff 5 is inflated to directly expand the stent 6 sleeved thereon, so that the two steps of placing the expanding catheter first and then the catheter sleeved with the stent in the prior art are completed in one step, the injury to the tissue is reduced and the medical efficiency is improved.

1. A medical catheter with a balloon (2) embedded in a head, comprising a catheter head (11), a catheter middle portion (12), a catheter tail (13); and
a balloon (2) which consists of a balloon connecting portion (21) and a balloon free portion (22);
wherein the catheter head (11) has a head recess (H110) for receiving the balloon free portion (22), and a head sidewall (H112) surrounding the head recess (H110);
the balloon connecting portion (21) is sealed to the catheter head (11), the balloon (2) has an inner surface (23), an outer surface (24), a balloon chamber area (1103) enclosed by the outer surface (24), and a farthest endpoint (A);
at least one part of the balloon free portion (22) is inserted into the head recess (H110), and the farthest endpoint (A) of the balloon free portion (22) is located in the head recess (H110);
a lower gap (1102) is defined between the inner surface (23) of the balloon free portion (22) and the head sidewall (H112), a lower gap fluid passage (1105) communicates with the lower gap (1102);
the balloon chamber area (1103), located in the head recess (H110) for containing a drug (D), is bag-like and has an opening (1106) toward an outside of the head recess (H110);
when an external fluid enters the lower gap fluid passage (1105) and then enters the lower gap (1102), a volume of the lower gap (1102) will increase, and the external fluid will squeeze the inner surface (23) to decrease a volume of the balloon chamber area (1103);
and, under the squeezing of the external fluid, the balloon chamber area (1103) disappears when the balloon free portion (22) completely bulges out from the head recess (H110) so as to release the drug (D) contained in the balloon chamber area (1103).

2. The medical catheter of claim 1, wherein the head sidewall (H112) is lobe-shaped, and the head sidewall (H112) is composed of at least two sidewall lobes (H112a) each having a top (H1121) and a bottom;
the top (H1121) of each sidewall lobe is free, while the bottom of each sidewall lobe is connected to the bottom of adjacent sidewall lobes; a sidewall lobe gap (H1120) is defined between two adjacent sidewall lobes (H112a);
and, the free top (H1121) of each sidewall lobe is located in the lower gap (1102) or outside the balloon (2).

3. The medical catheter of claim 1, further comprising an anti-adhesion mechanism (P)
the anti-adhesion mechanism (P) is for avoiding possible adhesion between the inner surface (23) of the balloon (2) and a surface (H111) of the head recess (H110), is a rib or a groove, which is disposed on the inner surface (23) of the balloon (2) and/or the head recess (H110).

4. The medical catheter of claim 1, wherein the balloon free portion (22) has a thin wall point (1107′) at which the balloon (2) is easy to be damaged by an external force.

5. The medical catheter of claim 1, further comprising a hollow elastic balloon (3) sleeved on the catheter tail (13) or the catheter middle portion (12) near the catheter tail (13),
the hollow elastic balloon (3) has an internal space (30) communicating with the lower gap fluid passage (1105), and fluid in the internal space (30) of the hollow elastic balloon (3) can be injected into the lower gap (1102) by squeezing the hollow elastic balloon (3).

6. The medical catheter of claim 1, wherein the balloon (2) has a plurality of balloon bumps (243) and/or balloon recesses (244) disposed on the outer surface (24) of the balloon (2).

7. The medical catheter of claim 1, wherein an elastic vinculum (20) having two ends is disposed in the head recess (H110), a first end of the elastic vinculum (20) is connected to the farthest endpoint (A) of the balloon free portion (22) or in a region adjacent to the farthest endpoint (A) of the balloon free portion (22), and a second end of the elastic vinculum (20) is connected to the catheter head (11) in the head recess (H110).

8. The medical catheter of claim 1, wherein the medical catheter (1) has an inner chamber (10), an inner opening (101) below the catheter head (11) and an outer opening (102) on the catheter tail (13);
both the inner opening (101) and the outer opening (102) communicate with the inner chamber (10) of the medical catheter (1).

9. The medical catheter of claim 1, further comprising a fixed cuff (F) sleeved on the medical catheter (1) below the catheter head (11).

10. The medical catheter of claim 1, further comprising an expanding cuff (4) having an inner chamber (41) sleeved on the medical catheter (1) below the catheter head (11), the medical catheter (1) has a catheter body and an expanding cuff fluid passage (40) in the catheter body, and the expanding cuff fluid passage (40) communicates with the inner chamber (41) of the expanding cuff (4);
when the expanding cuff (4) is disposed in a stenosis of a natural orifice that has been pre-expanded by the balloon (2), the expanding cuff (4) inflates after the external fluid is injected into the expanding cuff fluid passage (40) and enters into the inner chamber (41) of the expanding cuff (4), so as to expand corresponding biological tissue at the stenosis of the natural orifice.

11. The medical catheter of claim 1, further comprising a stent inflation cuff (5) having an inner chamber (51) sleeved on the medical catheter (1) below the catheter head (11), and a stent (6) sleeved on the stent inflation cuff (5), the medical catheter (1) has a catheter body and an stent inflation cuff fluid passage (50) in the catheter body, and the stent inflation cuff fluid passage (50) communicates with the inner chamber (51) of the stent inflation cuff (5);
when the stent inflation cuff (5) is disposed in a stenosis of a natural orifice that has been pre-expanded by the balloon (2), the stent inflation cuff (5) inflates after the external fluid is injected into the stent inflation cuff fluid passage (50) in the catheter body and enters into the inner chamber (51) of the stent inflation cuff (5), so as to expand the stent (6) and expand corresponding biological tissue at the stenosis.

12. The medical catheter of claim 1, further comprising a filling medium (S);
the filling medium(S) is pre-filled in the lower gap (1102) to partially or completely block contact between the inner surface (23) of the balloon (2) and the surface (H111) of the head recess (H110).
